(19) United States
(12) Patent Application Publication
(10) Pub. No.: US 2026/0026800 A1
(43) Pub. Date: Jan. 29, 2026

(54) SYSTEMS AND METHODS FOR PERFORMING TENSIONABLE KNOTLESS TISSUE REPAIRS

(71) Applicant: ARTHREX, INC., Naples, FL (US)

(72) Inventors: Andrew Christian Petry, JR., Naples, FL (US); Peter J. Dreyfuss, Naples, FL (US); Justin William Griffin, Virginia Beach, VA (US)

(21) Appl. No.: 19/272,281

(22) Filed: Jul. 17, 2025

Related U.S. Application Data
(60) Provisional application No. 63/674,389, filed on Jul. 23, 2024.

(51) Int. Cl. A61B 17/04

(52) U.S. Cl. CPC A61B 17/0487; A61B 17/0401; A61B 2017/0409; A61B 2017/0445; A61B 2017/0464

(57) ABSTRACT

Systems and methods are provided for performing tensionable knotless tissue repairs. A locking ferrule that includes a one-way locking mechanism may be utilized as part of the tensionable knotless tissue repairs for tensioning and locking one or more strands of suture. The one-way locking mechanism may be established by one or more locking barbs of the locking ferrule.

### CROSS-REFERENCE TO RELATED APPLICATIONS

This application claims the benefit of U.S. Provisional Application No. 63/674,389, which was filed on Jul. 23, 2024 and is incorporated herein by reference in its entirety.

### BACKGROUND

This disclosure relates to the field of surgery, and more particularly to systems and methods for performing a variety of tensionable knotless tissue repairs.

Repetitive trauma to a joint, such as a knee, ankle, hip, or shoulder joint, for example, may result in the development of tissue defects (e.g., soft tissue tears, cartilage defects, etc.). If not treated, tissue defects could further deteriorate, thereby causing joint instability and discomfort.

### SUMMARY

This disclosure relates to systems and methods for performing tensionable knotless tissue repairs. A locking ferrule that includes a one-way locking mechanism may be utilized during the tensionable knotless tissue repairs for tensioning and locking one or more strands of suture.

An exemplary surgical method may include, inter alia, attaching a graft to a tissue, loading a suture through the graft, implanting a suture anchor into a bone, shuttling the suture through the suture anchor, loading the suture through a cannulation of a locking ferrule, tensioning the suture in a first direction, and locking the suture within the cannulation to prevent movement of the suture in a second direction.

Another exemplary surgical method may include, inter alia, attaching a suture to a tissue, loading the suture through a surgical device, implanting the surgical device into a bone, loading the suture through a cannulation of a locking ferrule, inserting the locking ferrule into the bone, tensioning the suture in a first direction relative to the bone, and locking the suture within the cannulation to prevent movement of the suture in a second direction relative to the bone.

The embodiments, examples, and alternatives of the preceding paragraphs, the claims, or the following description and drawings, including any of their various aspects or respective individual features, may be taken independently or in any combination. Features described in connection with one embodiment are applicable to all embodiments, unless such features are incompatible.

The various features and advantages of this disclosure will become apparent to those skilled in the art from the following detailed description. The drawings that accompany the detailed description can be briefly described as follows.

### BRIEF DESCRIPTION OF THE DRAWINGS

FIG. 1 illustrates a locking ferrule that can be used for performing various tensionable knotless tissue repairs.

FIG. 2 is an end view of the locking ferrule of FIG. 1.

FIG. 3 is a cross-sectional view through section 3-3 of FIG. 1.

FIG. 4 is a cross-sectional view through section 4-4 of FIG. 2.

FIG. 5 schematically illustrates loading a suture through a locking ferrule.

FIG. 6 schematically illustrates locking a suture relative to a locking ferrule.

FIGS. 7, 8, 9, 10, 11, and 12 schematically illustrate a surgical method for performing a tissue repair.

FIGS. 13, 14, and 15 schematically illustrate another exemplary surgical method for performing a tissue repair.

### DETAILED DESCRIPTION

This disclosure relates to systems and methods for performing tensionable knotless tissue repairs. A locking ferrule that includes a one-way locking mechanism may be utilized during the tensionable knotless tissue repairs for tensioning and locking one or more strands of suture. These and other features of this disclosure are described in further detail below.

An exemplary surgical method may include, inter alia, attaching a graft to a tissue, loading a suture through the graft, implanting a suture anchor into a bone, shuttling the suture through the suture anchor, loading the suture through a cannulation of a locking ferrule, tensioning the suture in a first direction, and locking the suture within the cannulation to prevent movement of the suture in a second direction.

In any further embodiment, a locking barb of the locking ferrule locks the suture relative to a body of the locking ferrule.

In any further embodiment, the locking barb protrudes inwardly from an inner diameter wall of the locking ferrule.

In any further embodiment, the locking barb engages a thickened section of the suture to prevent its movement in the second direction.

In any further embodiment, loading the suture through the cannulation includes pulling the suture through the cannulation in the first direction with a suture loader.

In any further embodiment, the method includes moving an eyelet of the suture loader in the second direction through the cannulation prior to pulling the suture through the cannulation.

In any further embodiment, pulling the suture through the cannulation includes pulling a thinned section of the suture through the cannulation.

In any further embodiment, shuttling the suture through the suture anchor includes passing the suture through a proximal eyelet of a distal end portion of an anchor body of the suture anchor.

In any further embodiment, the surgical method includes positioning the locking ferrule within a cannulated passage of the suture anchor while tensioning the suture in the first direction.

In any further embodiment, the surgical method includes repairing a tear in the tissue prior to attaching the graft to the tissue.

In any further embodiment, attaching the graft to the tissue includes securing a medial side of the graft to the tissue.

In any further embodiment, the suture anchor is implanted at a location this is lateral to both an edge of the tissue and a lateral side of the graft.

In any further embodiment, the surgical method includes loading a second suture through the graft, implanting a second suture anchor into the bone, shuttling the second suture through the second suture anchor, loading the second suture through a second cannulation of a second locking ferrule, tensioning the second suture, and locking the second suture within the second cannulation to prevent movement of the second suture.

In any further embodiment, the surgical method includes positioning the second locking ferrule within a cannulated passage of the second suture anchor while tensioning the suture.

Another exemplary surgical method may include, inter alia, attaching a suture to a tissue, loading the suture through a surgical device, implanting the surgical device into a bone, loading the suture through a cannulation of a locking ferrule, inserting the locking ferrule into the bone, tensioning the suture in a first direction relative to the bone, and locking the suture within the cannulation to prevent movement of the suture in a second direction relative to the bone.

In any further embodiment, the locking ferrule is received over top of the surgical device within the bone.

In any further embodiment, the surgical device is a cortical button.

In any further embodiment, the cortical button is implanted within a tunnel formed in the bone.

In any further embodiment, the tunnel is a unicortical tunnel.

In any further embodiment, the tissue is a muscle.

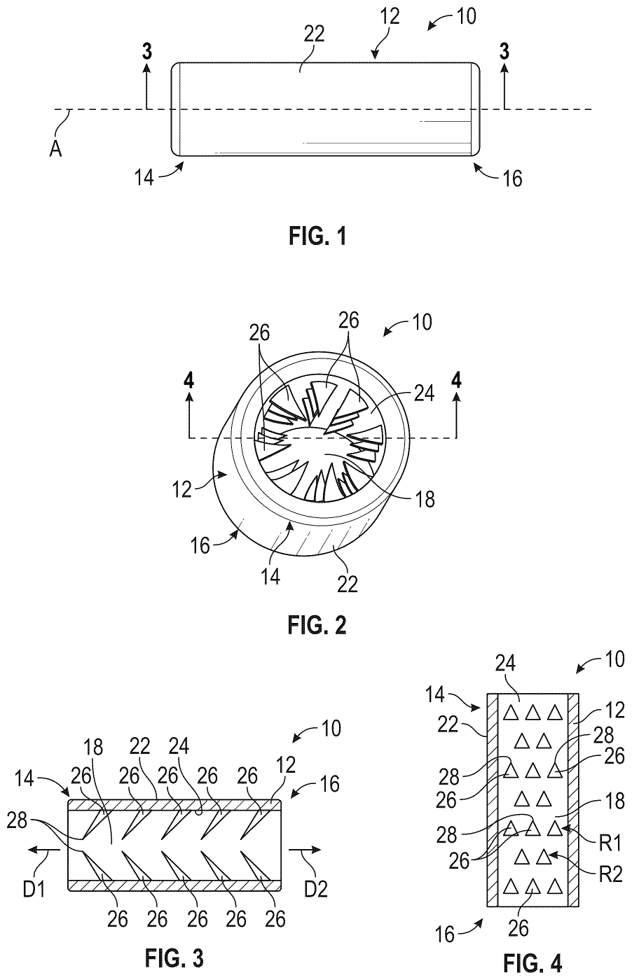

FIGS. 1-6 illustrate an exemplary locking ferrule 10 that can be used when performing various tensionable knotless tissue repairs. For example, the locking ferrule 10 could be utilized during surgical methods for attaching tissue (e.g., ligament, tendon, graft, etc.) to bone or for repairing any other type of tissue effect. The locking ferrule 10 could be used in conjunction with a variety of orthopedic surgical repairs, including but not limited to rotator cuff repairs, biceps tendon repairs, and subscapularis repairs, among many others.

The locking ferrule 10 may include a body 12 that extends along a longitudinal axis A between a proximal end 14 and a distal end 16. The body 12 may be tubular shaped and may be constructed from either metallic materials or plastic materials. However, the specific size, shape, and material make-up of the body 12 are not intended to limit this disclosure.

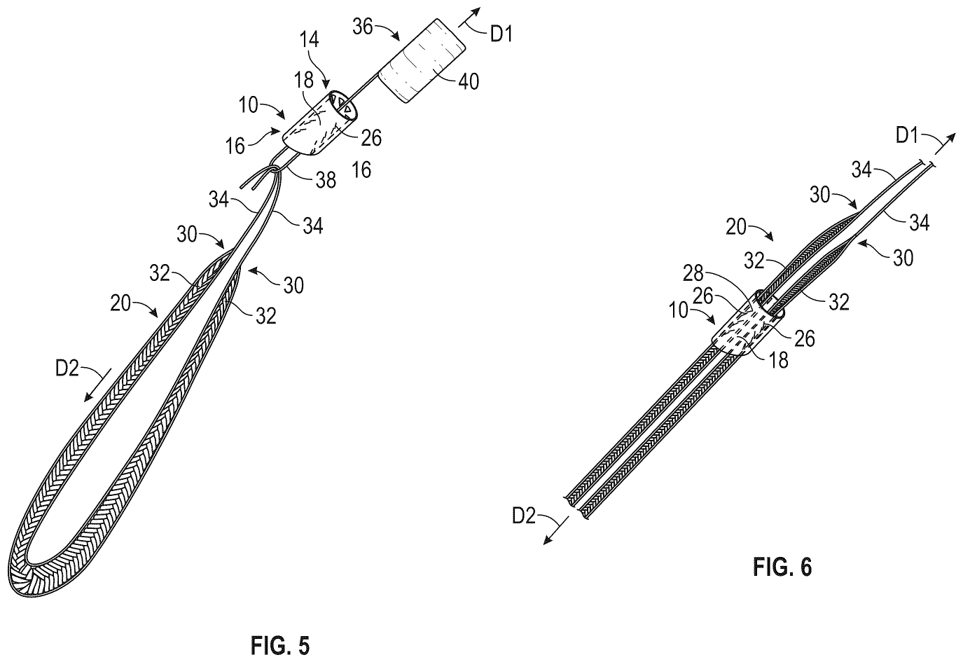

A cannulation 18 may extend through the body 12 and may establish an internal passageway for accommodating one or more strands of suture 20 (see, for example, FIGS. 5 and 6). The cannulation 18 may extend across an entire length of the body 12 and thus extends from the proximal end 14 to the distal end 16. The longitudinal axis A may bisect the cannulation 18.

The body 12 may include an outer diameter wall 22 and an inner diameter wall 24. The outer diameter wall 22 may be smooth or could alternatively include threads, barbs, or other features for facilitating fixation to bone. The inner diameter wall 24 may circumscribe the cannulation 18. In some embodiments, the cannulation 18 may taper in a direction toward the distal end 16 and is therefore narrower within the distal end 16 compared to within the proximal end 14.

A plurality of locking barbs 26 may protrude inwardly from the inner diameter wall 24. The locking barbs 26 may therefore occupy at least a portion of the open space of the cannulation 18. In an embodiment, the locking barbs 26 are integrally formed (e.g., molded) features of the body 12 of the locking ferrule 10. The locking barbs 26 may be provided along an entire length of the cannulation 18 or at only select portions thereof. The locking barbs 26 may be either rigid or flexible structures.

The locking barbs 26 may be arranged in multiple rows along the length of the cannulation 18. For example, the locking barbs 26 may be arranged in a least a first row R1 and a second row R2 (see FIG. 4). In an embodiment, the locking barbs 26 of the second row R2 are staggered relative to the locking barbs 26 of the first row R1 (see FIG. 4).

Each locking barb 26 may include a sharp or pointed tip 28, and each locking barb 26 may be angled in a common direction (e.g., here, toward the proximal end 14). The locking barbs 26 may therefore establish a one-way locking mechanism that permits one or more sutures 20 to pass through the cannulation in a first direction D1 while preventing the suture(s) 20 from being tensioned or otherwise moved back in a second direction D2. The staggered relationship of the rows of locking barbs 26 may provide for maximum engagement with the suture 20 one it is passed through the cannulation 18.

Referring now primarily to FIGS. 5 and 6, with continued reference to FIGS. 1-4, one or more sutures 20 may be passed through the cannulation 18. The suture 20 may be FiberWire®, FiberTape®, or any other suitable suture product. FiberWire® and FiberTape® are suture products marketed and sold by Arthrex, Inc. However, other suture products could be utilized for the suture 20 within the scope of this disclosure. The size and type of suture utilized in conjunction with the locking ferrule 10 are not intended to limit this disclosure.

The suture 20 may include a varying thickness. The suture 20 may therefore include one or more tapered regions 30 where the suture 20 transitions between a thickened section 32 and a thinned section 34. The thinned sections 34 can facilitate passing the suture 20 through the cannulation 18, such as via a suitable suture loader 36 that can be provided as part of a suture locking system that includes the locking ferrule 10, for example, and the thickened sections 32 can provide greater surface area for the locking barbs 26 to engage in order to sufficiently lock the suture 20 relative to the locking ferrule 10 after loading the suture 20 through the cannulation 18.

In an embodiment, the thickened sections 32 of the suture 20 are about twice as thick as the thinned sections 34. However, other ratios (e.g., 1.5:1, 3:1, etc.) between the relative thicknesses (e.g., outer diameters) of the thickened section 32 and the thinned section 34 of the suture 20 are contemplated within the scope of this disclosure.

The suture 20 may be passed through the cannulation 18 of the locking ferrule 10 using the suture loader 36. An eyelet 38 of the suture loader 36 may be passed through the cannulation 18 (e.g., by inserting the eyelet 38 at the proximal end 14 of the body 12 and then moving the suture loader 36 in the second direction D2). One or more thinned sections 34 of the suture 20 may then be loaded through the cyclet 38. The suture loader 36 may then be pulled, via a handle 40, in the first direction D1 to pass the suture 20 through the cannulation 18.

Once the suture 20 has been passed through the cannulation 18, the suture 20 may be further tensioned in the first direction D1 to lock the suture 20 relative to the locking ferrule 10. The locking barbs 26 substantially prevent the suture 20 from backing up or otherwise moving in the second direction D2. The pointed tips 28 of the locking barbs 26 may interdigitate with one or more of the thickened sections 32 to lock the suture 20 and prevent it from moving in the second direction D2.

Although shown as locking a single folded suture 20 in the above implementations, the locking ferrule 10 could be configured to receive and lock multiple strands of suture and/or sutures of varying sizes.

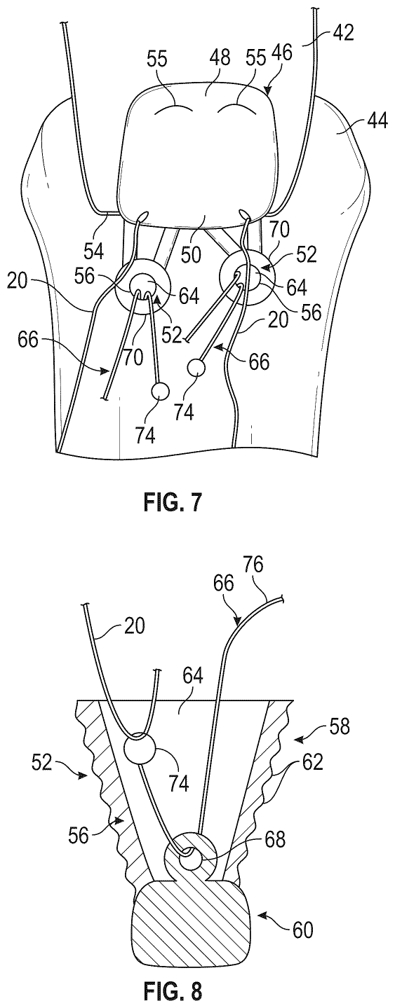

The locking ferrule 10 described above and shown in FIGS. 1-6 may be utilized to tension and knotlessly fixate one or more sutures as part of various surgical methods. FIGS. 7-12 schematically illustrate one such surgical method for attaching a graft 46 relative to a tissue 42 and a bone 44. The tissue 42 may have partially or completely torn away from the bone 44 during vigorous exercise or sporting activities, for example. When such tears occur, reattachment is often necessary to repair the tissue defect. The graft 46 may be secured over top of the tissue 42 in order to augment the repair. Notably, the various figures provided to schematically illustrate the method are not necessarily drawn to scale, and some features may be exaggerated or minimized to emphasize certain details of a particular component, assembly, or system.

The graft 46 could include either an allograft or an autograft. In an embodiment, the graft 46 is an acellular dermal extracellular matrix. ArthroFlex®, sold by Arthrex, Inc., is one type of graft suitable for use when performing the surgical method of FIGS. 7-12. In other implementations, the graft 46 could be a suture patch or any other type of synthetic scaffold.

The surgical method schematically illustrated in FIGS. 7-12 could be used in conjunction with a variety of orthopedic surgical repairs, including but not limited to rotator cuff repairs, for example. The bone 44 may therefore be associated with any joint of the human musculoskeletal system (e.g., shoulder, knee, hip, ankle, etc.).

In an embodiment, the surgical method is performed as an arthroscopic procedure by working through various arthroscopic portals. However, the exemplary surgical method could alternatively be performed as an open procedure within the scope of this disclosure. The exemplary surgical method may be employed to achieve secure fixation of the graft 46 after repairing or reconstructing the tissue 42 in a manner that provides mechanical strength and augments healing of the tissue 42.

Referring first to FIGS. 7 and 8, after optionally repairing the underlying tissue 42 and fixating a medial side 48 of the graft 46 to the tissue 42, such as with one or more mattress stitches 55, for example, the surgical method may include loading one or more sutures 20 through a lateral side 50 of the graft 46. The sutures 20 may be loaded through and connected to the graft 46 using any known stitching technique. In an alternative embodiment, the medial side 48 of the graft 46 may be fixated to the tissue using one or more medially placed fixation devices (e.g., suture anchors).

Next, a lateral row of fixation devices may be implanted into the bone 44. The lateral row of fixation devices may include one or more suture anchors 52. The suture anchors 52 of the lateral row may be placed laterally from an edge 54 of the tissue 42 and laterally from the lateral side 50 of the graft 46. However, other implantation locations could be selected based on the performing surgeon's preferences and depending on the type of orthopedic procedure being performed. Notably, although two suture anchors 52 are illustrated as being part of the lateral row in the illustrated embodiment, a greater or fewer number of lateral fixation devices could be utilized as part of the surgical method within the scope of this disclosure.

Each suture anchor 52 may include an anchor body 56 and a shuttle device 66 that is connected to the anchor body 56. The anchor body 56 may extend between a proximal end portion 58 and a distal end portion 60. The anchor body 56 may include a single piece or two-piece design, and thus the distal end portion 60 could be a separate piece that is attached to the proximal end portion 58. The distal end portion 60 may be blunt or could be sharp and thus configured to pierce or punch through tissue/bone.

The anchor body 56 may include a plurality of bone engagement features 62 that are designed to engage a bone wall when the suture anchor 52 in inserted into the bone 44. The bone engagement features 62 can include barbs, circular ledges, threads, or any other type of bone engagement feature or combination of features.

A cannulated passage 64 may extend at least partially through the anchor body 56. The shuttle device 66, which may be a passing wire or another suture, for example, may be accommodated within the cannulated passage 64 and may be connected to the anchor body 56. For example, the shuttle device 66 may be received through a proximal eyelet 68 of the distal end portion 60 for slidably connecting the shuttle device 66 relative to the anchor body 56. The proximal eyelet 68 may be at least partially accommodated within the cannulated passage 64.

The anchor body 56 may be made of a relatively rigid, biocompatible material, such as a biocomposite material or PEEK material, for example. However, other materials or combinations of material could be utilized to construct the anchor body 56 of each suture anchor 52.

The anchor body 56 of each suture anchor 52 may be inserted into a socket 70 formed in the bone 44. Each socket 70 may be a preformed opening formed in the bone 44 that is sized for receiving the anchor body 56 of one of the suture anchors 52. Alternatively, the socket 70 could be formed during insertion of the anchor body 56 into the bone 44.

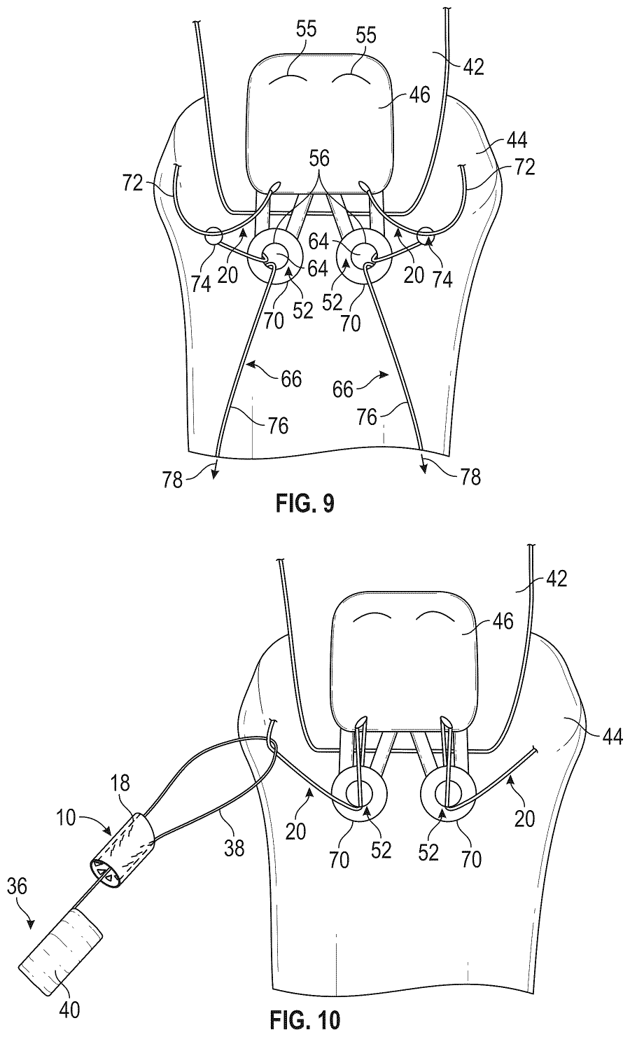

Referring to FIG. 9, the surgical method may proceed by shuttling the sutures 20 previously loaded through the lateral side of the graft 46 through the anchor bodies 56 of the suture anchors 52 of the lateral row. For example, a free end 72 of each suture 20 from the graft 46 may be passed through an eyelet 74 of the shuttle device 66 of a first of the suture anchors 52, and then a free end 76 of the shuttle device 66 may be pulled (e.g., in the direction of arrow 78) to allow the suture 20 to pass through the cannulated passage 64 and be accommodated through the proximal cyelet 68 of the distal end portion 60 of the anchor body 56, thereby operably connecting the suture 20 to the suture anchor 52. This suture shuttling process may be repeated to shuttle the free end 72 of another suture 20 from the graft 46 through the anchor body 56 of a second of the suture anchors 52 of the lateral row. Notably, the sutures 20 may be shuttled through the anchor bodies 56 either before or after implanting the anchor body 56 of each suture anchor 52 into its respective socket 70 in the bone 44.

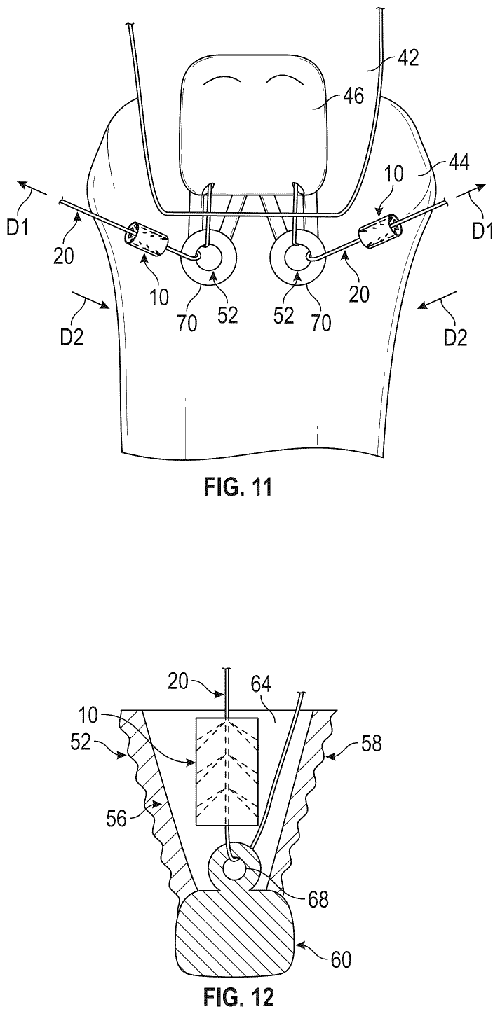

At this point of the surgical method, the sutures 20 from the graft 46 are preliminarily fixated relative to the bone 44 by the suture anchors 52. Each suture 20 may subsequently be further tensioned and locked in place using the locking ferrule 10. For example, as shown in FIGS. 10 and 11, after loading the suture 20 that is connected to the first of the suture anchors 52 through the cannulation 18 of the locking ferrule 10 using the suture loader 36, the suture 20 may be tensioned in the direction D1 to allow the locking ferrule 10 to slide down the suture 20 in the direction D2 toward the suture anchor 52. Further tension of the suture 20 in the direction D1 allows the pointed tips 28 of the locking barbs 26 to interdigitate with the suture 20 to lock the suture 20 in place and prevent it from sliding back in the second direction D2.

Once a sufficient amount of tension has been applied to the suture 20, the locking ferrule 10 may slide into engagement with the suture anchor 52. For example, the locking ferrule 10 may be accommodated within the cannulated passage 64 of the anchor body 56 of the suture anchor 52 (see FIG. 12).

The process shown in FIGS. 10-12 may be repeated to tension and lock the additional suture 20 associated with the second of the suture anchors 52 of the lateral row with an additional locking ferrule 10. The excess length of the sutures 20 that extend outside of the cannulations 18 of the locking ferrules 10 may be removed (e.g., cut) once tensioning and locking is complete.

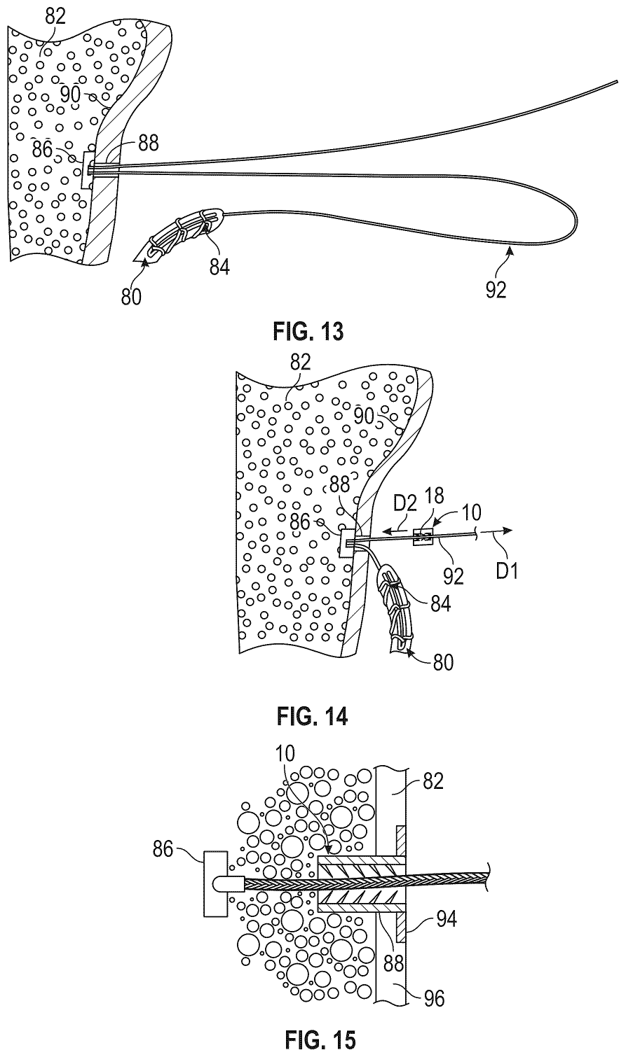

FIGS. 13-15 schematically illustrate another surgical method in which the locking ferrule 10 of FIGS. 1-6 may be utilized to tension and lock suture during a surgical repair. In this embodiment, the surgical method may involve securing a tissue 80 (e.g., muscle, tendon, etc.) to a bone 82, such as part of a subpectoral or suprapectoral biceps tenodesis procedure or a subscapularis repair, for example.

Referring first to FIG. 13, the surgical method may include creating a looping stitch 84 (e.g., with suture) within the tissue 80 and inserting a cortical button 86 within the bone 82 at a desired attachment point for the tissue 80. The cortical button 86 may be inserted into a tunnel 88 formed in the bone 82. The tunnel 88 may be a unicortical or bicortical tunnel formed in the bone 82 that is sized for receiving the cortical button 86. The cortical button 86 may be inserted into the tunnel 88 and then flipped to a position against a cortical shelf 90 of the bone 82. A suture limb 92 of the looping stitch 84 may be connected to the cortical button 86 prior to its insertion into the tunnel 88.

After flipping the cortical button, the tissue 80 may be reduced into place relative to the bone 82. The suture limb 92 may subsequently be tensioned and locked in place using the locking ferrule 10. For example, as shown in FIG. 14, after loading the suture limb 92 through the cannulation 18 of the locking ferrule 10, the suture limb 92 may be tensioned in the direction D1 to allow the locking ferrule 10 to slide in the direction D2 down and into the tunnel 88. Further tension of the suture limb 92 allows the pointed tips 28 of the locking barbs 26 to interdigitate with the section of the suture limb 92 accommodated within the cannulation 18, thereby locking the suture limb 92 in place and preventing it from sliding back in the second direction D2. The tissue 80 is therefore securely tensioned and fixated relative to the bone 82.

The locking ferrule 10 may be positioned within the tunnel 88 such that it overlies the cortical button 86 (see FIG. 15). In an exemplary implementation, the locking ferrule 10 may include a cap portion 94 that is configured to seat against an outer cortex 96 of the bone 82 to prevent over-insertion of the locking ferrule 10. The cap portion 94 may be an integrally formed feature of the locking ferrule 10, and when included, can provide a top hat-like appearance of the locking ferrule 10.

The locking ferrules of this disclosure may be utilized with one or more additional fixation devices (e.g., anchors, buttons, etc.) for performing various tensionable knotless tissue repairs. The locking ferrules provide for tensioning and retensioning suture(s) at various points of the tissue repair, including subsequent to implantation of the accompanying fixation devices, thus providing numerous advantages over prior tissue repair techniques.

Although the different non-limiting embodiments are illustrated as having specific components or steps, the embodiments of this disclosure are not limited to those particular combinations. It is possible to use some of the components or features from any of the non-limiting embodiments in combination with features or components from any of the other non-limiting embodiments.

It should be understood that like reference numerals identify corresponding or similar elements throughout the several drawings. It should further be understood that although a particular component arrangement is disclosed and illustrated in these exemplary embodiments, other arrangements could also benefit from the teachings of this disclosure.

The foregoing description shall be interpreted as illustrative and not in any limiting sense. A worker of ordinary skill in the art would understand that certain modifications could come within the scope of this disclosure. For these reasons, the following claims should be studied to determine the true scope and content of this disclosure.

What is claimed is:

1. A surgical method, comprising:
loading a suture through a graft;
implanting a suture anchor into a bone;
shuttling the suture through the suture anchor;
loading the suture through a cannulation of a locking ferrule;
tensioning the suture in a first direction; and
locking the suture within the cannulation to prevent movement of the suture in a second direction.

2. The surgical method as recited in claim 1, wherein a locking barb of the locking ferrule locks the suture relative to a body of the locking ferrule.

3. The surgical method as recited in claim 2, wherein the locking barb protrudes inwardly from an inner diameter wall of the locking ferrule.

4. The surgical method as recited in claim 2, wherein the locking barb engages a thickened section of the suture to prevent its movement in the second direction.

5. The surgical method as recited in claim 1, wherein loading the suture through the cannulation includes:
pulling the suture through the cannulation in the first direction with a suture loader.

6. The surgical method as recited in claim 5, comprising:
moving an eyelet of the suture loader in the second direction through the cannulation prior to pulling the suture through the cannulation.

7. The surgical method as recited in claim 5, wherein pulling the suture through the cannulation includes:
pulling a thinned section of the suture through the cannulation.

8. The surgical method as recited in claim 1, wherein shuttling the suture through the suture anchor includes:
passing the suture through a proximal eyelet of a distal end portion of an anchor body of the suture anchor.

9. The surgical method as recited in claim 1, comprising:
positioning the locking ferrule within a cannulated passage of the suture anchor while tensioning the suture in the first direction.

10. The surgical method as recited in claim 1, comprising:
repairing a tear in a tissue prior to attaching the graft to the tissue.

11. The surgical method as recited in claim 1, comprising, prior to loading the suture through the graft:
attaching the graft to a tissue.

12. The surgical method as recited in claim 1, wherein the suture anchor is implanted at a location this is lateral to both an edge of a tissue and a lateral side of the graft.

13. The surgical method as recited in claim 1, comprising:
loading a second suture through the graft;
implanting a second suture anchor into the bone;
shuttling the second suture through the second suture anchor;
loading the second suture through a second cannulation of a second locking ferrule;
tensioning the second suture; and
locking the second suture within the second cannulation to prevent movement of the second suture.

14. The surgical method as recited in claim 13, comprising:
positioning the second locking ferrule within a cannulated passage of the second suture anchor while tensioning the suture.

15. A surgical method, comprising:
attaching a suture to a tissue;
loading the suture through a surgical device;
implanting the surgical device into a bone;
loading the suture through a cannulation of a locking ferrule;
inserting the locking ferrule into the bone;
tensioning the suture in a first direction relative to the bone; and
locking the suture within the cannulation to prevent movement of the suture in a second direction relative to the bone.

16. The surgical method as recited in claim 15, wherein the locking ferrule is received over top of the surgical device within the bone.

17. The surgical method as recited in claim 15, wherein the surgical device is a cortical button.

18. The surgical method as recited in claim 17, wherein the cortical button is implanted within a tunnel formed in the bone.

19. The surgical method as recited in claim 18, wherein the tunnel is a unicortical tunnel.

20. The surgical method as recited in claim 15, wherein the tissue is a muscle.
